(19) United States
(12) Patent Application Publication
(10) Pub. No.: US 2026/0076740 A1
(43) Pub. Date: Mar. 19, 2026

(54) PULSATING LASER AND METHOD OF CONTROL THEREOF

(71) Applicant: LASER TEAM MEDICAL LTD., Katzrin (IL)

(72) Inventors: Alon SHACHAM, Katzir (IL); Rotem NAHEAR, Kiryat Arba (IL); Neria SULIMAN, Jerusalem (IL); Salman NOACH, Jerusalem (IL)

(73) Assignee: LASER TEAM MEDICAL LTD., Katzrin (IL)

(21) Appl. No.: 19/151,385

(22) PCT Filed: Jan. 25, 2024
(86) PCT No.: PCT/IL2024/050100

§ 371 Date: Jul. 28, 2025

Related U.S. Application Data
(60) Provisional application No. 63/441,185, filed on Jan. 26, 2023.

(51) Int. Cl. A61B 18/22; A61B 17/00; A61B 18/00

(52) U.S. Cl. CPC A61B 18/22; A61B 2017/00154; A61B 2018/00577; A61B 2018/00589; A61B 2018/2222

(57) ABSTRACT

A laser ablation system is disclosed. The laser system comprises a laser cavity; a gain medium; a pump; a scanner; and a controller configured to: receive, a system pulse frequency; receive a desired pulse frequency; calculate, based on the system pulse frequency and the desired pulse frequency, a number of points to be included in a grid wherein each point corresponds to a location of a pulse; receive at least one of: a size of a treatment area, a shape of the treatment area, and the required pulse density; determine the number of grids required for covering the treatment area and locations of the pulses in each grid based on the received size of the treatment area, shape of the treatment area, and the required pulse density; and control the pump and the scanner to provide laser pulse to all locations in each grid.

### CROSS-REFERENCE TO RELATED APPLICATIONS

This application claims the benefit of priority of U.S. Provisional Patent Application No. 63/441,185, titled “PULSATING LASER AND METHOD OF CONTROL THEREOF”, filed Jan. 26, 2023. The contents of all the above applications are incorporated herein by reference in their entirety.

### FIELD OF THE INVENTION

Exemplary embodiments of the present invention relate generally to pulsating lasers. More specifically, the present invention relates to controlling pulsating lasers to achieve a desired ablation in multiple locations with controllable coagulation diameter.

### BACKGROUND OF THE INVENTION

Laser systems are widely used in medical fields, for example, to perform precise surgeries or any dermal intervention. The benefits of laser systems are their capability of producing a laser beam with a high energy output focused on a miniscule, precise location. Current laser systems used in the art may include active or passive Q-switchers, which are used to create laser pulses at the nanosecond scale.

In certain medical procedures, for example, cosmetic laser, a laser is used to create small ablations spots on a large area of the skin. The ablation must be as precise as possible, homogeneous, and should cover relatively large (e.g., several cm²) areas. Therefore, each one of the following parameters must be controlled, the location of each spot, the width of each spot, and the depth of each spot.

Accordingly, there is a need for a laser system capable of producing controlled ablation scanning over relatively large tissue areas.

### SUMMARY OF THE INVENTION

Embodiments of the present invention are directed to a laser ablation system comprising: a laser cavity; a gain medium disposed within the laser cavity configured to produce a pulsating laser beam at a specific wavelength; a pump configured to optically pump a lasing medium; a scanner configured to control the movement of the laser beam; and a controller. In some embodiments, the controller is configured to: receive, a system pulse frequency; receive a desired pulse frequency; calculate, based on the system pulse frequency and the desired pulse frequency, a number of points to be included in a grid wherein each point corresponds to a location of a pulse; receive at least one of: a size of a treatment area, a shape of the treatment area, and the required pulse density; determine the number of grids required for covering the treatment area and locations of the pulses in each grid based on the received size of the treatment area, shape of the treatment area, and the required pulse density; and control the pump and the scanner to provide laser pulse to all locations in each grid.

In some embodiments, the controller is further configured to receive, from a user interface, a desired coagulation diameter at each location, and to determine the desired pulse frequency based on the desired coagulation diameter. In some embodiments, the controller is further configured to: receive a number N of pulses required to be provided to each location; for each grid, determine a sequence of pulse provision locations in the grid; and repeat the sequence N times. In some embodiments, the controller is further configured to receive, from a user interface, a desired ablation depth and to determine N based on the desired ablation depth. In some embodiments, at least some of the grids have different N.

In some embodiments, receiving a system pulse frequency comprises one of: receiving the system pulse frequency from a database and measuring the system pulse frequency using an energy meter. In some embodiments, calculating the number of points to be included in a grid is by dividing the system pulse frequency by the desired pulse frequency.

In some embodiments, if determining the number of grids required for covering the treatment area is not an integer, the method may include adjusting at least one of: the size of the treatment area, the system pulse frequency and the desired pulse frequency. In some embodiments, adjusting the desired pulse frequency comprises selecting the pulse frequency from a range of pulse frequencies. In some embodiments, adjusting the size of the treatment area comprises providing to a user a selection of optional treatment areas each corresponding to a multiplication of the grid area with an integer. In some embodiments, adjusting the system pulse frequency is by changing a current provided to the laser pump.

Some additional aspects of the invention are directed to a method of controlling a laser ablation system, comprising: receiving the laser ablation system pulse frequency; receiving a desired pulse frequency; calculating based on the system pulse frequency and the desired pulse frequency, a number of points to be included in a grid wherein each point corresponds to a location of a pulse; receiving at least one of: a size of a treatment area, a shape of the treatment area, and the required pulse density; determining the number of grids required for covering the treatment area and locations of the pulses in each grid based on the received size of the treatment area, a shape of the treatment area, and the required pulse density; and controlling a pump configured to optically pump a lasing medium, and a scanner configured to control the movement of the laser pulses, to provide laser pulses to all locations in each grid.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram of a laser ablation system, according to some embodiments of the invention;

FIG. 1B is a block diagram of a laser cavity, according to some embodiments of the invention;

FIG. 1C is a block diagram of a computing device for a laser system according to some embodiments of the invention;

FIG. 2 is a flowchart of a method of controlling a laser ablation system according to some embodiments of the invention;

FIGS. 3A and 3B are illustrations of grids of laser spots in an area to be treated by the laser ablation system according to some embodiments of the invention; and

FIG. 4 is a flowchart of another method of controlling a laser ablation system according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Disclosed herein is a laser system, e.g. a laser operating in the 2 micrometers (“μm”) wavelength range, for example, 1.8-2.2 μm, 1.85-2.2 μm, 1.85-2.05 μm, 1.85-2.0 μm, 1.9-2.1 μm, or any value in between. In some embodiments, the laser is a pulsed laser, which may be used for a variety of applications, e.g. surgery, for any type of tissue ablation and/or coagulation, cosmetics applications, military applications, material processing, optical communication, LIDAR or the like.

In some embodiments, the laser system disclosed herein may comprise a q-switching element to control a laser beam of the laser system. As used herein the term “q-switching element” may refer to either a passive q-switch (also referred to as “saturable absorber (SA)”) or an active q-switch. In some embodiments, a q-switching element may control the laser beam, for example, to modulate a pulse of the laser beam. In a non-limiting example, a q-switching element may be comprised of a saturable absorber (SA), which may passively absorb a laser beam in order to produce a pulsed laser beam at a desired energy, further discussed herein below.

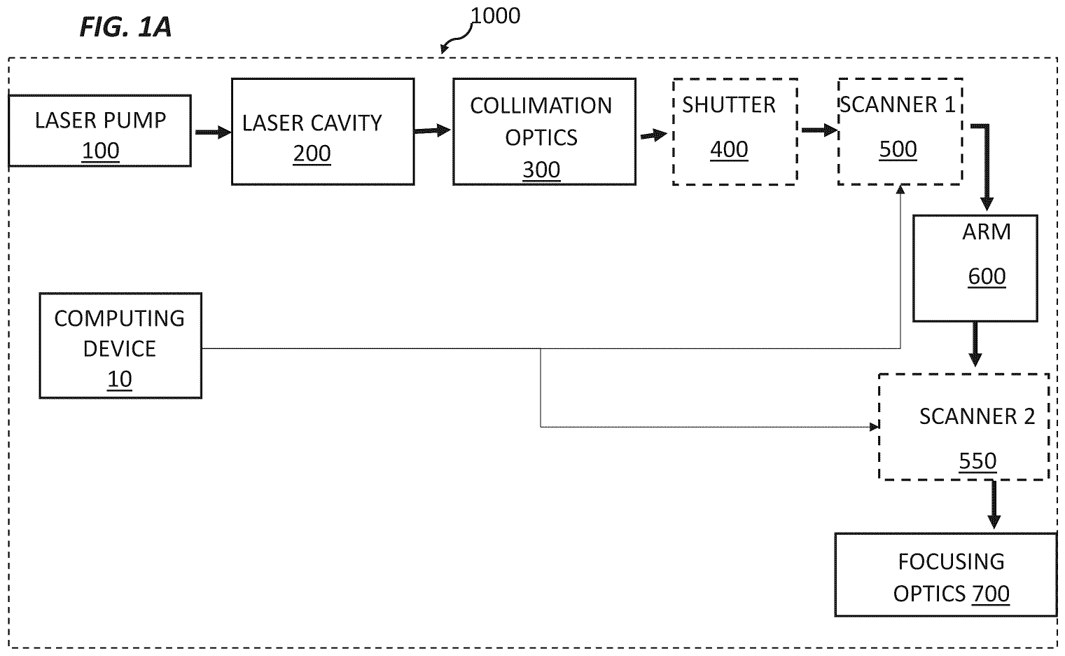

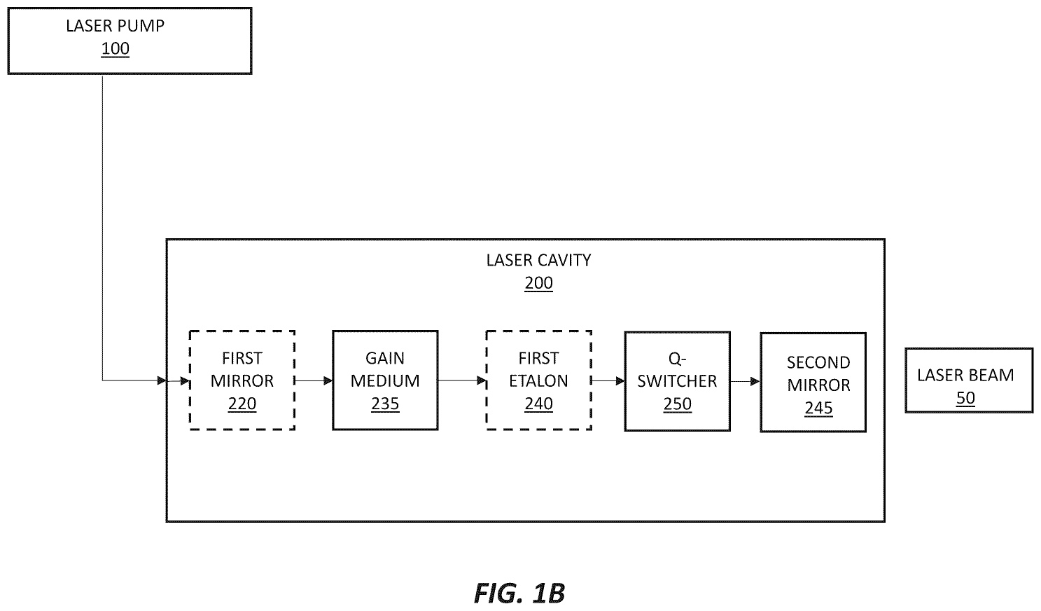

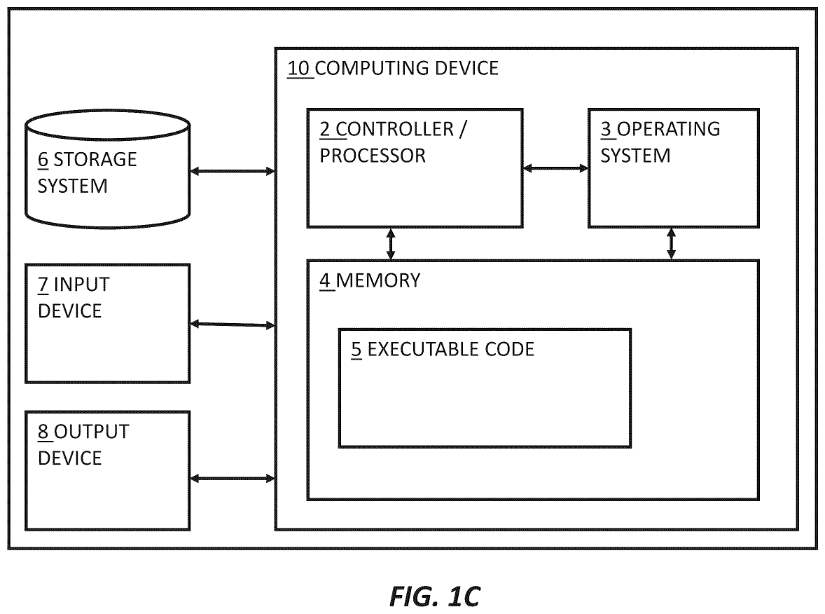

Reference is now made to FIG. 1A is a block diagram of a laser ablation system, according to some embodiments of the invention. According to some aspects, a laser system 1000 may comprise a pump 100 (such as a pump diode) configured to optically pump a lasing medium. In some embodiments, pump 110 for providing a laser beam to laser cavity 200, discussed in detail with respect to FIG. 1B.

In some embodiments, pump 100 may be tuned to provide a beam having a wavelength which matches the corresponding absorption peaks of a gain medium as described below. There are various pump schemes and pumping configurations well-known in the art and some of them may be applied to the present disclosure application. In some embodiments, pump diode 100 may include direct pumping. In some embodiments, the pump diode 100 configurations may include a side pump and an end pump.

In some embodiments, from cavity 200 the laser beam (e.g., beam 50 illustrated in FIG. 1B) is directed to a collimation optics 300. In some embodiments, the collimation optic is a converging lens. In some embodiments, the focal length of the lens will be determined based on scanner 500 and arm 600 restrictions on the laser beam diameter. For example, in the case of scanner 500 mirrors diameter is 10 mm the optimal laser beam diameter shall not exceed 6 mm. In the case of laser beam 50 with 6 mrad half angle the collimation optic 300 focal lengths cannot exceed 500 mm

In some embodiments, system 1000 may further include a shutter 400 that may be any optical shutter. In some embodiments, the shutter blocks the laser beam by mechanical means. For example, the shutter-blocking mechanism may consist of a single blade or diaphragm. Shutter 400 may be configured to control the provision of the laser beam. In some embodiments, system 1000 may not include a shutter.

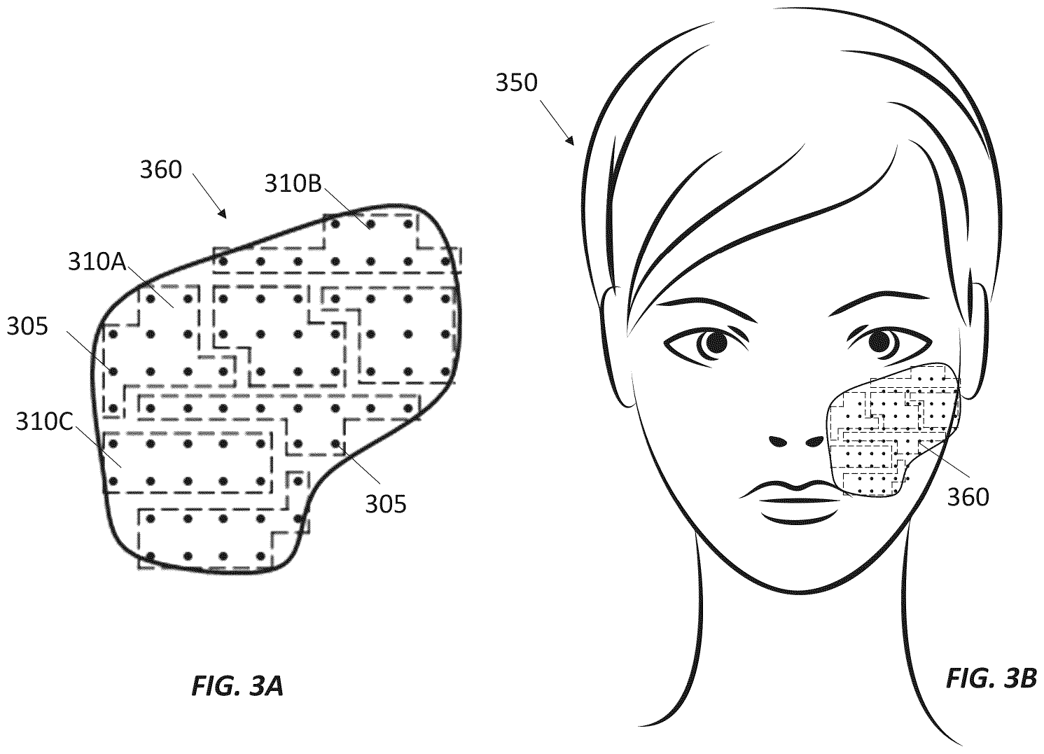

In some embodiments, system 100 includes at least one scanner 500 and/or scanner 550. Scanner 500 and/or scanner 550 may allow an automatic movement of the laser beam from one location to the other. For example, scanner 500 and/or scanner 550 may allow scanning of a pulsating laser beam from one point on a grid to the other point according to a scanning scheme illustrated and discussed with respect to FIGS. 3A and 3B.

In some embodiments, scanner 500 may be located after shutter 400 or directly after collimation optics 300. Alternatively, scanner 550 may be located after an arm 600 prior to focusing optics 700.

Some nonlimiting examples for, scanner 500 and/or scanner 550 may include galvanometer mirror scanners (galvo scanners) piezoelectric fast steering mirrors, voice coil-driven steering mirrors, motorized kinematic mirror mounts, and motorized gimbal mounts.

In some embodiments, arm 600 may include a set of two or more pivotally connected hollow tubes configured to deliver and manipulate the direction of the laser beam. Arm 600 may include a set of mirrors, prism, or any other required optical element that may allow a direct manipulation of the laser beam. Arm 600 may be operated by a user (e.g., a professional) in order to direct the laser beam towards the area to be treated, for example, a cheek of a user, as illustrated in FIG. 3B. In some embodiments arm 600 may include relay optic to maintain laser beam 50 properties over the length of the arm. In some embodiments, the relay optics in the arm will convert angular movement from scanner 500 to focusing optics 700 while confining laser beam 50 inside the arm.

In some embodiments, system 1000 may further include focusing optics 700 that may include mirrors converging and diverging lenses and diffractive elements or any combination of them.

In some embodiments, all the controllable components of system 1000 may be controlled by a computing device 10 illustrated and discussed with respect to FIG. 1C. Computing device 10 may control at least laser pump 100, scanner 500, and/or scanner 550 to provide a pulsating laser beam that automatically scans an area, for example, of a skin of a user.

Reference is now made to FIG. 1B which is a block diagram of a laser cavity 200 according to some embodiments of the invention. In some embodiments, laser pump 100 may provide a laser beam to a first end of a laser cavity 200.

In some embodiments, laser pump 100 may provide a pump beam to a gain medium 235, for example, via mirror 220. Non-limiting exemplary gain media 235 are selected from materials (also referred to as “laser crystals”) doped with a rare-earth element. In some embodiments, the material is a crystal selected from Yttrium Aluminum Garnet (“YAG”), Yttrium Lithium Fluoride (“YLF”), and Yttrium Aluminum Phosphorus (“YAP”). In some embodiments, the rare earth element is selected from Thulium (Tm), Holmium (Ho), Erbium (Er), or any combination thereof.

Further non-limiting exemplary gain media 235 are selected from: Tm:YAG, Tm:YVO₄, Tm:YLF, Tm:YAP or Tm:LuAG. In some embodiments, the concentration of the Tm³⁺ dopant in the host crystal material of the laser crystal is inversely proportional to the length of the laser crystal. In some embodiments, the concentration of Tm³⁺ dopant is between about 0.2 wt. % to about 8 wt. % and any value in between, for example, 1-6 wt. %, 2-7 wt. %, 2-8, 0.5 to 7 wt. % and the like.

In some embodiments, gain medium 235 may be disposed within a laser cavity 200.

In some embodiments, laser cavity 200 may include q-switching element 250 In some embodiments, q-switching element 250 may be disposed within a laser cavity 200.

In some embodiments, the q-switching element 250 may be in the form of a thin layer or film. In some embodiments, non-limiting exemplary q-switching element 140 may comprise a material selected from doped ZnS crystals, and doped ZnSe crystals e.g., chromium doped ZnSe crystals, chromium doped ZnS crystals, Ho doped materials, such as, Ho:YAG, Ho:YLF, Ho:YAP Ho doped fiber or a combination thereof. In some embodiments, further non-limiting exemplary q-switching element 140 may comprise a material selected from doped silver halide or a chalcogenide.

In some cases, the Cr:ZnSe and the Chromium doped Zinc Sulfide (“Cr:ZnS”) SA may have a relatively high absorption cross-sections, thus not requiring a focusing mode to a small area on the SA. This may provide more flexibility with respect of the resonator. In some embodiments, the Cr:ZnSe and the Cr:ZnS saturable absorbers are capable of a low saturable intensity, which may lead to reduced risk of damage during Q-switched operation.

In some exemplary embodiments of the subject matter, the Cr:ZnS crystal saturable absorber may be applied in several passive Q-switch (“PQS”) lasers, e.g. Ho:YAG, Tm:KY(WO₄), Tm:KLu(WO₄), or the like. In some cases, the SA may fulfill a passive Q-switch when

$${σ}_{S A} / {A}_{S A} > {σ}_{g} / {A}_{g} ,$$

where σ_{SA} and σ_{g} represent the absorption cross section of the saturable absorber and the emissions cross section of a gain medium at the lasing wavelength, respectively, and A_{SA} and A_{g} may be the mode area at the saturable absorber and gain medium.

In some embodiments, gain (lasing) medium 235, and q-switching element 250, which are disposed along the same longitudinal axis.

In some embodiments, laser cavity 200 may further comprise: a first mirror 220, first etalon 240, and second mirror 245.

In some embodiments, the radius of curvature of the concave\convex first mirror 220 may be in the −50 to plano and 30 to plano respectively and any value in between, for example, −100 to plano and 50 to plano, −200 to plano and 100 to plano and the like. In some embodiments, the first mirror 220 may be positioned in a light-path e.g., approximately along the longitudinal axis of the laser cavity 200.

In some embodiments, first etalon 240 is paired with a second etalon (not illustrated) further disposed within laser cavity 200. In some embodiments, at least one first or second etalon may be configured to provide tunability of laser beam with respect to a spectral range of laser beam 50.

In some embodiments, the second etalon is positioned next to the first etalon 240, so as the first and the second etalons are positioned a light-path of the laser beam.

In some embodiments, the etalons 240A and 240B are positioned along a horizontal axis 290 comprising gain medium 235. In some embodiments, horizontal axis 290 may be defined as up to ±60 degrees from a longitudinal axis.

In some embodiments, etalons 240A and 240B, provide a tunable spectral range and a narrow spectral bandwidth of the laser. In some embodiments, the transmission wavelength band of the laser light is dictated by reflectivity, thickness, and refractive index of etalons 240A and 240B, and thus a pulse width thereof is adjusted. In some embodiments, the tunability range is at least 10 nm, at least 14 nm, at least 20 nm, at least 25 nm, at least 30 nm, at least 35 nm. In some embodiments, the tunability range is from 8 to 50 nm, or, in some embodiments from 8 to 15 nm, or, in some embodiments, from 10 to 15 nm, or, in some embodiments from 15 to 20 nm, or, in some embodiments, from 20 to 30 nm, or, in some embodiments from 30 to 35 nm, or, in some embodiments from 35 to 40 nm.

In some embodiments, the tunability range may depend on the gain medium 235. In some embodiments, the tunability range may depend on reflectance degree of the output coupler, and/or transmission degree of the q-switching element.

In some embodiments, the first etalon is thinner than the second etalon.

In some embodiments, the thickness ratio of the first etalon 240 to the second etalon is from 1:5 to 1:40, respectively. In some embodiments, a thickness ratio of etalon 240A to etalon 240B is 1:5, 1:10, 1:15, 1:20, 1:25, 1:30, 1:35, or 1:40, respectively, including any value and range therebetween.

Without being bound by any particular theory or mechanism, it is assumed that the thinner etalon allows tunability of the spectral range. In some embodiments, a thinner etalon thickness provides a wider tunability of the spectral range. Further, and without being bound by any particular theory or mechanism, it is assumed that the thicker etalon response for the spectral bandwidth narrowing, with the maximum thickness being limited to avoid the occurrence of two spatial adjacent modes. In some embodiments, the use of two etalons provides both features of spectral range tunability and spectral bandwidth narrowing.

In some embodiments, the first and second etalons may comprise a full or a partial reflecting material e.g., in the form of a coating.

In some embodiments, laser cavity 200 may have a Q-switching element 250, which may allow operation of the laser system 1000 in pulsed mode. In some embodiments, Q-switching element 250 may be a passive Q-switching element, or alternatively, an active Q-switching element.

In some embodiments, active Q-switching element 250 may be, an optical modulation unit, in some embodiments, positioned within a resonator. In some embodiments, an acousto-optic modulator (AOM), an electro-optic modulator (EOM), or an acousto-optic tunable filter (AOTF) may be included as an optical modulator in the optical modulation unit.

In some embodiments, laser cavity 200 may have a Q-switching element such as acousto-optic modulator (AOM). In some embodiments, AOM may be positioned in a light-path of the laser beam e.g., proximately along the longitudinal axis of the laser cavity 200.

In some embodiments, the AOM may be configured to receive and modulate a seed laser beam. In some embodiments, the laser beam may be arranged to be generally incident at the Bragg angle to the AOM. In some embodiments, the AOM may allow to produce a pulsed output beam. In some embodiments, the AOM may control the timing of the release of the pulse from the seed laser.

In another configuration, laser system 200 comprises a passive Q-switching element 250 instead of the AOM. In some embodiments, passive Q-switching element may be configured to provide passive pulse switching of the laser beam. In some embodiments, a non-limiting example of a passive Q-switching element 250 is a saturable absorber (SA). In some embodiments, as laser system 200 provides passive Q-switching element 250 with a laser a short pulse laser beam may be produced. In some embodiments, non-limiting exemplary q-switching element 250 may comprise a material selected from doped ZnS crystals, and doped ZnSe crystals e.g., chromium doped ZnSe crystals, chromium doped ZnS crystals, Ho doped materials, such as, Ho:YAG, Ho:YLF, Ho:YAP Ho doped fiber or a combination thereof.

In some embodiments, SA comprises a semiconductor. In some embodiments, SA comprises a quantum dot. In some embodiments, SA comprises a doped crystal. In some embodiments, non-limiting exemplary doped crystals are selected from: chromium (II) doped zinc selenide (Cr:ZnSe) and chromium (II) doped zinc sulfide (Cr:ZnS). In some embodiments, the w/w (weight per weight) concentration of Cr²⁺ dopant in a doped crystal is between about 1% to about 20%, or in some embodiments, from 9 to 13%. In some cases, Cr:ZnSe and Cr:ZnS SA may have a relatively high absorption cross-sections, thus not requiring a focusing mode to a small area on the SA. In some embodiments, this may provide more flexibility with respect of the resonator. In some embodiments, the Cr:ZnSe and the Cr:ZnS SA have a low saturable intensity, which may lead to reduced risk of damage during Q-switched operation. In some embodiments, the Cr:ZnS crystal SA may be applied in several passive Q-switch (“PQS”) lasers, e.g. Ho:YAG, Tm:KY(WO₄), Tm:KLu(WO₄), or the like.

In some embodiments, laser beam 50 may be emitted at a second end of laser cavity 200. In some embodiments, laser beam 50 may be a pulsed laser according to some aspects of the invention. In some embodiments, laser beam 50 may be characterized by pulse energy and frequency and laser wavelength as a result of controlling laser pump 100 or first etalon 240.

Reference is now made to FIG. 1C, which is a block diagram depicting a computing device, which may be included within an embodiment of laser ablation system 1000, according to some embodiments of the present invention.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. Although, for the sake of clarity, a single item of executable code 5 is shown in FIG. 1C, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data related to correlations (e.g., a lookup table) between a frequency of a laser beam and a diameter of the tissue ablation, and a lasing power of the laser beam based on an ablation depth may be stored in storage system 6 and may be loaded from storage system 6 into memory 4 where it may be processed by processor or controller 2. In some embodiments, some of the components shown in FIG. 1C may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 10 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

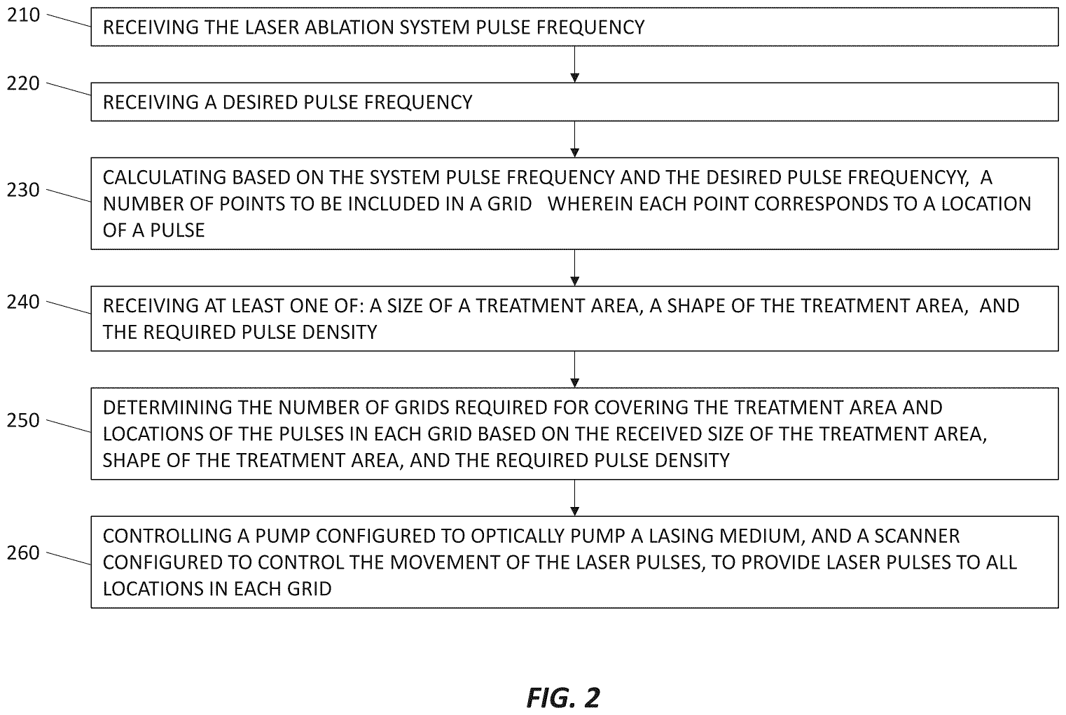

Reference is now made to FIG. 2, which is a flowchart of a method of controlling a laser ablation system (e.g., system 1000) according to some embodiments of the invention.

The method of FIG. 2 may be executed by controller 2 of computing device 10 or by any other suitable controller.

In step 210, the controller may receive a system pulse frequency, for example, from a database associated with system 1000, or from a user via a user interface such as, input device 7. The system pulse frequency is the frequency of beam 50 exiting laser cavity 200, and depends mainly on q-switching element 250. In some embodiments, receiving a system pulse frequency comprises one of: receiving the system pulse frequency from a database and measuring the system pulse frequency using an energy meter.

In step 220, the controller may receive a desired pulse frequency. In some embodiments, the desired pulse frequency may be calculated based on a desired coagulation diameter at each location. For example, the desired pulses frequency may be determined based on a pulse's energy (a parameter of system 1000) and the desired coagulation diameter. For example, a look up table or look up tables comprising the frequency desired coagulation diameter may be stored in a memory, such as, storage system 6. Nonlimiting examples for such tables are given and discussed below with respect to Table 1. In the nonlimiting example of Table 1, if a coagulation diameter of 100 μm is required the pluses are provided at 50 Hz.

Table 1 herein below details an exemplary resulting diameter (width) output of a laser beam, according to some embodiments of the invention, based on a frequency input of the device:

TABLE 1

| Desired Pulses frequency (Hz) | Width (μm) |
|---|---|
| 10 | 10-40 |
| 50 | 70-100 |
| 600 | >120 |

In step 230, the controller may calculate, based on the system pulse frequency and the desired pulse frequency a number of points to be included in a grid wherein each point corresponds to a location of a pulse. In some embodiments, the number of points to be included in a grid may be determined based on equation 1.

$$n = {f}_{s} / {f}_{d} \qquad ( 1 )$$

Wherein, n is the number of points to be included in a grid, fₛ the system pulse frequency, and f_{d} the desired pulse frequency.

In step 240, the controller may receive at least one of: a size of a treatment area, a shape of the treatment area, and the required pulse density. For example, controller 2 may receive from a user (e.g., a professional) via input 7, the size and shape of treatment area 360 illustrated in FIGS. 3A and 3B. In yet another example, controller 2 may receive from a user (e.g., a professional) via input 7 the required pulse density in order to achieve a desired treatment result (e.g., deep piling, brightening, etc.) In some embodiments, the area of each grid is determined based on n and the distance between each to neighboring points 305 (e.g., locations of the provide pulses on the tissue) which can be a parameter stored in storage system 6 or received from the user.

In step 250, the controller may determine the number of grids required for covering the treatment area and locations of the pulses in each grid based on the received size of the treatment area, shape of the treatment area, and the required pulse density. For example, the controller may. For example, in the nonlimiting example, of FIGS. 3A and 3B, in order to cover area 360 grids 310A, 310B, 310C, etc. may be required.

In some embodiments, if the determining the number of grids required for covering the treatment area in not an integer, the controller may adjust at least one of: the size of the treatment area, the system pulse frequency, and the desired pulse frequency. For example, if the size of the treatment area is equal to 6¼ times the areas of a grid, controller 2 may reduce the treatment area to be exactly 6 times the areas of a grid or increase the treatment area to be exactly 7 times the areas of a grid. In some embodiments, adjusting the size of the treatment area comprises providing to a user with a selection of optional treatment areas each corresponding to a multiplication of the grid area with an integer.

In yet another example, controller 2 may reduce fₛ (the system pulse frequency) and or increase/decrease fa (the desired pulse frequency) thus reducing n which will result in reduce area of the grid allowing the treatment area to be exactly 7 times the areas of the new grid. In some embodiments, adjusting the desired pulse frequency comprises selecting the pulse frequency from a range of pulse frequencies or by changing the desired frequency up to a predetermined percentage. In some embodiments, adjusting the system pulse frequency is by changing a current provided to the laser pump.

In step 260, the controller may control the pump to optically pump a lasing medium, and the scanner is configured to control the movement of the laser pulses, to provide laser pulses to all locations in each grid. For example, controller 2 may control laser pump 100 to optically pump a lasing medium and further control scanner 500 and/or scanner 550 to move that pulsating laser from one location to the other in each grid. In some embodiments, the controller is further configured to: receive a number N of pulses required to be provided to each location; for each grid, determine a sequence of pulse provision locations in the grid; and repeat the sequence N times. In some embodiments, controller 2 is further configured to receive, from a user interface (input 7), a desired ablation depth and to determine N based on the desired ablation depth. In some embodiments, the ablation depth determined the amount of energy needs to be provided at leach location, as shown, in table 2,

| E_{T} = Energy (mJ) | Depth (mm) |
|---|---|
| 50 | 0.2-0.4 |
| 200 | 0.7-1.1 |
| 400 | 1-1.4 |
| 800 | 1.1-1.7 |
| 1600 | 1.6-2 |

N may be determined using equation 2.

$$N = {E}_{T} / {E}_{p} \qquad ( 2 )$$

Wherein, Er is the total energy required to be provided in each location, and Ep the energy of each pulse. In a non-limiting example, for a desired ablation with a depth of 1 mm the required total energy is 400 mJ, therefore, if the pulse energy is 10 mJ, the number of pulses is N=40. Therefore, controller 2 may control scanner 500 and/or scanner 550 to scan the entire grid with the pulsating laser 40 times. In some embodiments, each scan may start at a starting location and end at an ending location. In some embodiments, the ending location may or may not be adjacent to the starting location. Controller 2 may repeat each scan N times.

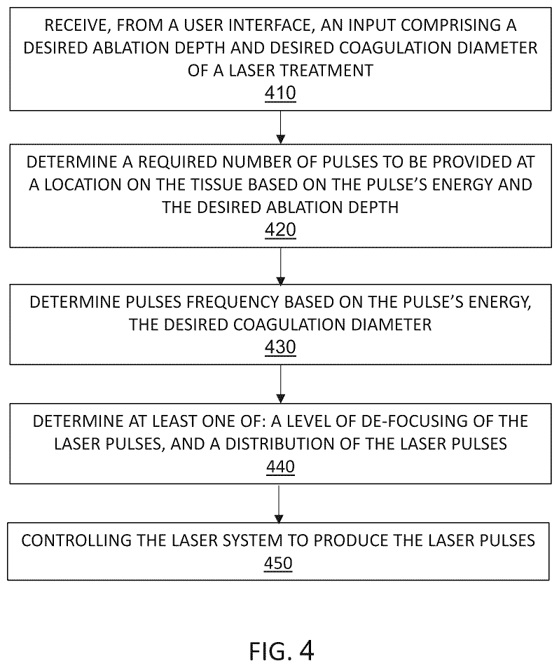

Reference is now made to FIG. 4 which flowchart of another method of controlling a laser ablation system according to some embodiments of the invention. In some embodiments, steps 410 to 450 may be used to control laser system 1000. In some embodiments, steps 410 to 450 may be controlled by controller 10 of laser system 1000 or any other suitable controller.

In step 410, an input may be received from a user interface, by controller thereof, comprising a desired ablation depth and desired coagulation diameter of a laser treatment. In some embodiments, the user interface may be associated or included in controller 10, for example the user interface may include any one of input devices 7 and output devices 8.

In step 420, a required number of pulses to be provided at a location on the tissue may be determined based on the pulse's energy and the desired ablation depth. For example, a look up table or look up tables comprising the required number of pulses associated with the desired ablation depth may be stored in a memory, such as, storage system 6. Nonlimiting examples for such a table are given and discussed herein with respect to Table 1. As the amount of energy provided by each pulse is known, e.g., 1 mJ, 1.2 mJ, 2 mJ, 3 mJ, 5 mJ and the like, the controller may calculate the number of required pulses. In the nonlimiting example of Table 1, in order to ablate to a depth of 1 mm, 100 mJ are required. Accordingly, if each laser pulse provides 4 mJ, 25 pulses will be required to ablate a depth of 1 mm.

In step 430, a required pulse frequency may be determined based on a pulse's energy and the desired coagulation diameter. For example, a look up table or look up tables comprising the frequency desired coagulation diameter may be stored in a memory, such as, storage system 6. Nonlimiting examples for such tables are given and discussed herein with respect to Table 2. In the nonlimiting example of Table 2, if a coagulation diameter of 150 μm is required the pluses are provided at 10 Hz.

In step 440, controller 10 may determine at least one of: a level of de-focusing of the laser pulses, and a distribution of the laser pulses. For example, in order to have a larger ablation diameter, controller 10 may manipulate the laser pulse by at least tow ways. In a first example, the system may cause slight defocusing of the beam, artificially broadening the beam's diameter. In a nonlimiting example, the defocusing may cause enlargement of the diameter by between 5 to 100 μm. In a second example, scanner 500 may cause minor shifting of the focal point, on the treated tissue, of some of the provided pulses, for example, shifting of 5-100 μm. In nonlimiting example, 50 out of 100 provided pulses may be shifted from the original location 305 of the provided pulses on the tissue, illustrated in FIG. 3A.

In step 550, a laser system or controller thereof may control the laser system to to produce the laser pulses at the determined number of pulses, pulses frequency and at least one of: the level of de-focusing of the laser pulses, and the distribution of the laser pulses.

In some embodiments, the laser system may be controlled to emit a pulsed laser, wherein a duration of the pulse is between 2 to 100 nanoseconds, 2 to 20 nanoseconds, 20 to 50 nanoseconds, 50 to 100 nanoseconds and any range and value herein between. In some embodiments, the laser beam produced may be characterized by a wavelength of 2 μm regime. In some embodiments, the laser beam may be characterized by energy (also referred to as “lasing power”) of at least 1 mJ, 1 to 15 mJ, 1 to 5 mJ, 5 to 15 mJ and any range and value herein between.

In some embodiments, an electrical power may be supplied to the laser system, in order to produce said laser beam, wherein said electrical power may be between 1 to 50 Watts, 1 to 16 Watts, 10 to 30 Watts, 25 to 50 Watts and any range and value herein between.

In some embodiments, computing device 10 may determine a first set of parameters comprising a first number of pulses, a first pulses frequency and at least one of: a first level of de-focusing of the laser pulses and a first distribution of the laser pulses; and a second set of parameters comprising a second number of pulses, a second pulses frequency and at least one of: a second level of de-focusing of the laser pulses and a second distribution of the laser pulses. In some embodiments, computing device 10 may further control the laser system to produce the laser pulses using the first set following by the second set.

In some embodiments, computing device 10 may determine sets with additional parameters and may control the laser system to produce the laser pulses according to any number of sets. As should be understood by the one skilled in the art, the invention is not limited to two sets, and any number of sets equal to or lager than two is within the scope of the invention. In some embodiments, at least some of the sets may be identical while others may differ by at least one parameter. In some embodiments, computing device 10 may control the laser system to produce laser pulses at alternating sets of parameters.

In some embodiments, each set of parameters may include only the number of pulses, and the pulses frequency, and the sets of parameters may differ from each other by at least one of the number of pulses, and the pulses frequency. In some embodiments, at least some of the sets may further include the level of de-focusing of the laser pulses and the distribution of the laser pulses.

A nonlimiting example may include, creating 0.5 mm depth with 20 μm coagulation treatment followed by 0.5 mm depth with 200 μm wide coagulation to create a clean epidermal layer with extensive dermal coagulation. In some embodiments, the input from the user may be either explicit, meaning stating all of the parameters for all the relevant treatments, or it may be preprogrammed to include only some of the parameters. For example, the first set may include a fixed width and frequency (e.g., an initial constant treatment) and the second set may be the selected operation mode in all treatment provided, and the parameters. In such an example, the overall depth may be the result of first and second sets and treatments, and the coagulation width the result of the second set.

In some embodiments, the laser beam may be focused into a Raman gain crystal. In some embodiments, the laser system may emit a spot diameter size of a laser beam between 20 to 200 microns, 20 to 40 microns, 40 to 100 microns, 100 to 200 microns and any range and value herein between.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments have been presented. Each of these embodiments may of course include features from other embodiments presented, and embodiments not specifically described may include various features described herein.

1. A laser ablation system comprising:
a laser cavity;
a gain medium disposed within the laser cavity configured to produce a pulsating laser beam at a specific wavelength;
a pump configured to optically pump a lasing medium;
a scanner configured to control the movement of the laser beam; and
a controller, configured to:
receive, a system pulse frequency;
receive a desired pulse frequency;
calculate, based on the system pulse frequency and the desired pulse frequency, a number of points to be included in a grid wherein each point corresponds to a location of a pulse;
receive at least one of: a size of a treatment area, a shape of the treatment area, and the required pulse density;
determine the number of grids required for covering the treatment area and locations of the pulses in each grid based on the received size of the treatment area, shape of the treatment area, and the required pulse density; and
control the pump and the scanner to provide laser pulse to all locations in each grid.

2. The laser ablation system of claim 1, wherein the controller is further configured to receive, from a user interface, a desired coagulation diameter at each location, and to determine the desired pulse frequency based on the desired coagulation diameter.

3. The laser ablation system of claim 1, wherein the controller is further configured to:
receive a number N of pulses required to be provided to each location;
for each grid, determine a sequence of pulse provision locations in the grid; and
repeat the sequence N times.

4. The laser ablation system of claim 3, wherein the controller is further configured to receive, from a user interface, a desired ablation depth and to determine N based on the desired ablation depth.

5. The laser ablation system of claim 3, wherein at least some of the grids have different N.

6. The laser ablation system of claim 1, wherein receiving a system pulse frequency comprises one of: receiving the system pulse frequency from a database and measuring the system pulse frequency using an energy meter.

7. The laser ablation system of claim 1, wherein calculating the number of points to be included in a grid is by dividing the system pulse frequency by the desired pulse frequency.

8. The laser ablation system of claim 1, wherein if the determining the number of grids required for covering the treatment area in not an integer, adjusting at least one of: the size of the treatment area, the system pulse frequency and the desired pulse frequency.

9. The laser ablation system of claim 8, wherein adjusting the desired pulse frequency comprises selecting the pulse frequency from a range of pulse frequencies.

10. The laser ablation system of claim 8, wherein adjusting the size of the treatment area comprises providing to a user a selection of optional treatment areas each corresponding to a multiplication of the grid area with an integer.

11. The laser ablation system of claim 8, wherein adjusting the system pulse frequency is by changing a current provided to the laser pump.

12. A method of controlling a laser ablation system, comprising:
receiving the laser ablation system pulse frequency;
receiving a desired pulse frequency;
calculating based on the system pulse frequency and the desired pulse frequency, a number of points to be included in a grid wherein each point corresponds to a location of a pulse;
receiving at least one of: a size of a treatment area, a shape of the treatment area, and the required pulse density;
determining the number of grids required for covering the treatment area and locations of the pulses in each grid based on the received size of the treatment area, shape of the treatment area, and the required pulse density; and
controlling a pump configured to optically pump a lasing medium, and a scanner configured to control the movement of the laser pulses, to provide laser pulses to all locations in each grid.

13. (canceled)

14. (canceled)

15. (canceled)

16. (canceled)

17. (canceled)

18. (canceled)

19. (canceled)

20. (canceled)

21. (canceled)

22. (canceled)
